(19) United States
(12) Patent Application Publication
(10) Pub. No.: US 2023/0086651 A1
(43) Pub. Date: Mar. 23, 2023

(54) CONTENT DELIVERY TECHNIQUES FOR CONTROLLING BIOMETRIC PARAMETERS

(71) Applicant: Oura Health Oy, Oulu (FI)

(72) Inventors: Jason Russell, Hamilton, MA (US); Chris Becherer, San Francisco, CA (US); Lauren Maymar, Belvedere, CA (US); Mats Kyyrö, Oulu (FI); Laura Furman, San Francisco, CA (US); Dmitrii Aleksandrov, Vantaa (FI); Cristian David Díaz, Helsinki (FI); Morgan Hecht, Brooklyn, NY (US); Ashley Hopkins, Brooklyn, NY (US)

(21) Appl. No.: 17/950,183

(22) Filed: Sep. 22, 2022

Related U.S. Application Data
(60) Provisional application No. 63/247,361, filed on Sep. 23, 2021.

(51) Int. Cl. G06F 16/9535; G16H 20/70; G16H 20/30; A61B 5/00

(52) U.S. Cl. CPC G06F 16/9535; A61B 5/0002; G16H 20/30; G16H 20/70

(57) ABSTRACT

Methods, systems, and devices for content delivery are described. A device may receive biometric data associated with a user from a wearable device. The device may determine that a biometric parameter of a set of biometric parameters associated with the biometric data satisfies a threshold during an occasion. The device may select media content from a set of media content for recommending to the user. Each respective media content of the set of media content may be scored based on a respective effectiveness associated with each respective media content for controlling a value of the biometric parameter. The selecting may be triggered based on the biometric parameter satisfying the threshold. The media content may be selected based on a score associated with the media content. The device may output the media content via a graphical user interface (GUI) of the apparatus during the occasion.

### CROSS REFERENCE

The present Application for Patent claims the benefit of U.S. Provisional Pat. Application No. 63/247,361 by RUSSELL et al., entitled “CONTENT DELIVERY TECHNIQUES FOR CHANGING BIOMETRIC PARAMETERS,” filed Sep. 23, 2021, assigned to the assignee hereof, and expressly incorporated by reference herein.

### FIELD OF TECHNOLOGY

The following relates to wearable devices and data processing, including content delivery techniques for controlling (e.g., changing, maintaining) biometric parameters.

### BACKGROUND

Some devices may be configured to provide content recommendations relevant to users. These devices may provide the content recommendations using some techniques, such as collaborative filtering and content-based filtering. In some cases, these devices may use collaborative filtering to recommend content based on a community of users and their preferences. Alternatively, in some other cases, these devices may use content-based filtering to recommend content based on information extracted from the content. However, these conventional techniques implemented by these devices are deficient.

### BRIEF DESCRIPTION OF THE DRAWINGS

FIGS. 1 and 2 illustrate examples of systems that support content delivery techniques for controlling biometric parameters in accordance with aspects of the present disclosure.

FIGS. 3 and 4 illustrate examples of graphical user interfaces (GUIs) that support content delivery techniques for controlling biometric parameters in accordance with aspects of the present disclosure.

FIG. 5 shows a block diagram of a device that supports content delivery techniques for controlling biometric parameters in accordance with aspects of the present disclosure.

FIG. 6 shows a block diagram of a wearable application that supports content delivery techniques for controlling biometric parameters in accordance with aspects of the present disclosure.

FIG. 7 shows a diagram of a system including a device that supports content delivery techniques for controlling biometric parameters in accordance with aspects of the present disclosure.

FIGS. 8 through 12 show flowcharts illustrating methods that support content delivery techniques for controlling biometric parameters in accordance with aspects of the present disclosure.

### DETAILED DESCRIPTION

Applications may be configured to recommend various forms of media content (e.g., audio content, video content, etc.) to users based on user preferences, previous selections by the user, or preferences inferred from groups of users. An application associated with health and wellness tracking may include audio content, video content, and the like. For example, a wellness application may include several audio files for guided mediations or video files related to exercise routines. Existing techniques for recommending media content to a user based on their previous selections or their general preferences may fail to identify and recommend the specific content that is most effective in causing one or more physiological responses for that particular person. For example, even though the user may have previously selected or may prefer a first type of meditation, it may be the case that a different type of meditation is more effective at lowering that user’s heart rate, respiration rate, or causing some other desirable physiological response. As such, improvements to existing techniques of content recommendation, especially in the context of content that is intended to have a physiological, mental, or other health impact on a user, are needed.

A system including a wearable device and a user device may collect biometric data (also referred to as physiological data), and based on the collected biometric data may deliver media content recommendations to the user. For example, the collected biometric data may indicate that the user has a sleep issue, such as a sleep latency (e.g., a timeframe when the user typically transitions to a sleep state, such as a rapid eye movement (REM) sleep state or a non-REM sleep state). Based on the identified sleep issue, the system may recommend a media content that may help the user transition to the sleep state earlier (e.g., reduce the user’s sleep latency). While the sleep latency serves as an example of biometric data, various other forms of biometric data or physiological insights derived from measured data may be used as triggers to deliver or recommend media content to a user.

For example, the collected biometric data may indicate a heart rate of the user while the user is engaged in an activity (e.g., running). Based on the identified heart rate of the user and the activity the user is engaged in, the system may recommend a media content that may help the user maintain their current heart rate within some threshold range. For instance, if the system identifies that the user’s heart rate has decreased below a threshold heart rate range, the system may retrieve media content that may help increase the user’s heart rate (e.g., music with a faster beat). Conversely, if the system identifies that the user’s heart rate has increased above the threshold heart rate range, the system may retrieve media content that may help decrease the user’s heart rate (e.g., music with a slower beat). In some cases, the heart rate range (e.g., threshold heart rate) may be input by the user, automatically determined by the system based on the type of activity (e.g., running), determined by the system based on the user’s activity/fitness/training goals, and the like.

Additionally, or alternatively, the collected biometric data may indicate a change in the heart rate of the user while the user is engaged in the activity (e.g., running). Based on the identified heart rate of the user and the activity the user is engaged in, the system may recommend a different media content that may help the user increase or decrease the user’s heart rate, for example, to a target heart rate or baseline heart rate associated with the activity.

In some implementations, the system may set or define thresholds for a biometric parameter (e.g., a health metric) of a set of biometric parameters associated with biometric data, where the pre-defined thresholds may trigger media content recommendations or other actions when the respective thresholds are satisfied. For example, if the system determines that a biometric parameter for a user satisfies (e.g., exceeds) a pre-defined threshold, the system may automatically trigger media content recommendation to the user. In some implementations, thresholds and/or ranges for a biometric parameter may be set or defined by a user to trigger media content recommendations or other actions when the respective thresholds set or defined by the user are satisfied. As noted previously, by way of example, if a user’s sleep latency satisfies a pre-defined threshold (e.g., the user is taking longer than 20 minutes to transition to a sleep state), the system may trigger media content recommendation to deliver a media content that may help the user transition to the sleep state earlier (e.g., reduce the user’s sleep latency). In some other examples, if a user’s heart rate satisfies a pre-defined threshold and there’s no activity detected (e.g., movement) by the user that would correlate to the user’s heart rate, the system may trigger media content recommendations to deliver a media content that may help lower the user’s heart rate (e.g., reduce stress).

In some implementations, the system may provide media content recommendations based on an input from a user. For example, if a user wants to be more energetic, the system may retrieve media content recommendations for the user that are configured to change (e.g., increase or decrease) a biometric parameter of the user, such as a heart rate, in order to help make the user more energetic. In some other implementations, the system may provide media content recommendations based on a community of users (e.g., a group of users associated with an application for a wearable device) and their respective aggregate biometrics. By way of example, the system may analyze collected biometric data from the user and compare them to the aggregate biometrics of the community and media content recommendations provided to these users. The system may also analyze the effectiveness of the media content to the biometrics, and based on the effectiveness of the media content, recommend the media content to the user. As used herein, the effectiveness of media content may be referred to as any measurable correlation between a user’s interaction with a piece of media content (e.g., listening to a meditation, watching a video, etc.), and a change in a measurable physiological parameter (e.g., heart rate, skin temperature, respiration rate, sleep metrics, readiness metrics, etc.) as described in accordance with various examples herein. As noted previously, by way of example, the system may analyze sleep latencies for the community of users and media content recommended to the community of users. The system may analyze the effectiveness of the recommended media content as an aggregate for improving sleep latency for the community of users. As such, if certain media content effectively reduced sleep latency or maintained a sleep latency for the community of users, the system may recommend this media content to the user. This technique may be used, for example, if a particular user has not yet interacted with the media content of an application, and the system has not yet collected biometric feedback from that user in relation to interacting with the media content.

The system may select and provide media content recommendations to the user based on a respective score of the media content. The score may indicate a respective effectiveness for inducing a change to, or to maintain, a value of at least one biometric parameter associated with biometric data of the user. In some implementations, the system may rank media content according to its relative score for inducing a change to, or to maintain, a value of at least one biometric parameter. Different media content may have different scores based on having different effectiveness for changing or maintaining certain biometric parameters. For example, a media content affecting sleep latency for a user may have a different score compared to another media content affecting a heart rate for the user. Additionally, or alternatively, two or more media contents that are associated with the same biometric parameter (e.g., a sleep latency) may have different scores (e.g., based on their relative effectiveness as compared to each other).

As a result, the system facilitates improvements to the user’s biometrics by providing targeted media content recommendations in real-time based on the user’s biometrics. While much of the present disclosure is described in the context of biometrics, this is not to be regarded as a limitation of the present disclosure. In particular, techniques described herein may enable media content recommendation to a user that may help improve the biometrics. Moreover, biometric data associated with a user may be used to update any score, measure, metric, or other abstraction associated with a user’s health or activity.

Aspects of the disclosure are initially described in the context of systems supporting physiological data collection from users via wearable devices. Additional aspects of the disclosure are described in the context of example GUIs. Aspects of the disclosure are further illustrated by and described with reference to apparatus diagrams, system diagrams, and flowcharts that relate to content delivery techniques for controlling biometric parameters.

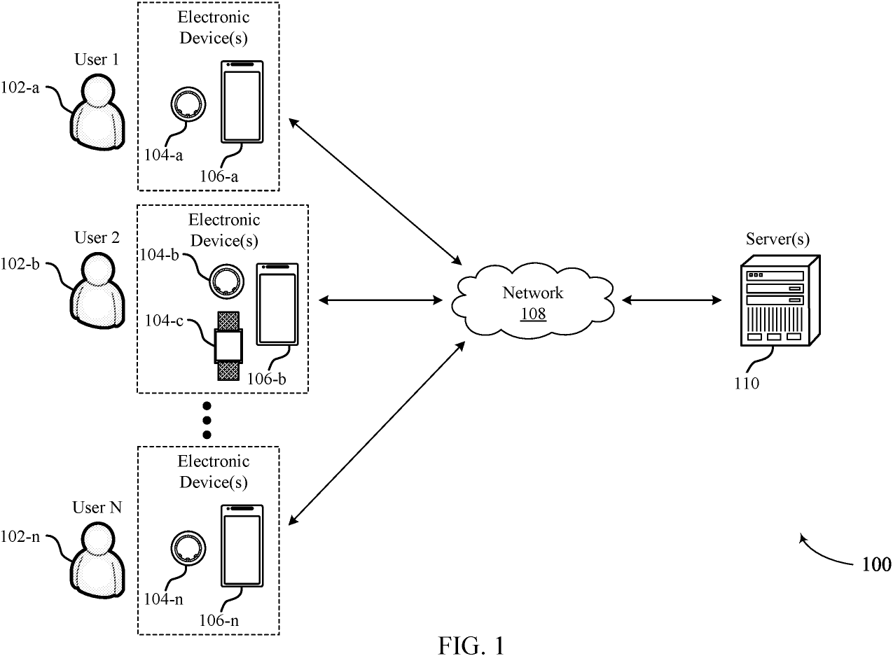

FIG. 1 illustrates an example of a system 100 that supports content delivery techniques for controlling biometric parameters in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user’s 102 finger, a wrist computing device (e.g., a smart watch, fitness band, or bracelet) configured to be worn on a user’s 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some examples, user devices 106 may include handheld mobile computing devices, such as smartphones and tablet computing devices. The user devices 106 may also include personal computers, such as laptop and desktop computing devices. Other examples of the user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices that may measure physiological parameters and some may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some examples, the wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user’s finger. In particular, a ring 104 may utilize one or more LEDs (e.g., red LEDs, green LEDs) that may emit light on the palm-side of a user’s finger to collect physiological data based on arterial blood flow within the user’s finger. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some examples, the system 100 may detect periods of time when a user 102 is asleep, and classify periods of time when the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some examples, data collected by the ring 104-a may be input to a machine learning classifier, where the machine learning classifier is configured to determine periods of time when the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user’s sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some examples, the system 100 may utilize circadian rhythm-derived features to further improve physiological data collection, data processing procedures, and other techniques described herein. The term circadian rhythm may refer to a natural, internal process that regulates an individual’s sleep-wake cycle that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user’s natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some examples, the system 100 may utilize other biological rhythms to further improve physiological data collection, analysis, and processing by phase of these other rhythms. For example, if a weekly rhythm is detected within an individual’s baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some examples, the respective devices of the system 100 may support techniques for content delivery for controlling biometrics. In particular, the system 100 illustrated in FIG. 1 may support techniques for recommending media content to a user 102 by causing a user device 106 corresponding to the user 102 to display an indication of the recommended media content. For example, as shown in FIG. 1, User 1 (user 102-a) may be associated with a wearable device 104-a (e.g., ring 104-a) and a user device 106-a. In this example, the ring 104-a may collect biometric data associated with the user 102-a, including heart rate, respiratory rate, skin temperature, and the like. In some examples, biometric data collected by the ring 104-a may be used to select the media content (e.g., music, audio book, podcast, video, etc.) for recommending to the user 102. The selection of the media content may be performed by any of the components of the system 100, including the ring 104-a, the user device 106-a associated with User 1, the one or more servers 110, or any combination thereof. Upon selecting the media content (e.g., music, audio book, podcast, video, etc.) for recommending to the user 102, the system 100 may selectively cause the GUI of the user device 106-a to display an indication of the recommended media content.

In some implementations, upon receiving the biometric data, the system 100 may determine that at least one biometric parameter associated with the biometric data satisfies a threshold during an occasion. For example, upon receiving the biometric data associated with the user 102-a from the ring 104-a, any of the components of the system 100, including the ring 104-a, the user device 106-a associated with User 1, the one or more servers 110, or any combination thereof, may determine that at least one biometric parameter, such as a heart rate, a respiratory rate, or the like satisfies a threshold during a time interval. Any of the components of the system 100, including the ring 104-a, the user device 106-a associated with User 1, the one or more servers 110, or any combination thereof, may then select media content from a set of media content for recommending to the user 102-a. In some other implementations, any of the components of the system 100, including the ring 104-a, the user device 106-a associated with User 1, the one or more servers 110, or any combination thereof, may select media content from a set of media content for recommending to the user 102-a after a period from the collected biometrics (e.g., sleep latency, amount of REM or deep sleep, etc.).

In some cases, any of the components of the system 100, including the ring 104-a, the user device 106-a associated with User 1, the one or more servers 110, or any combination thereof, may score each respective media content of the set of media content (e.g., music, audio book, podcast, video, etc.) based on a respective effectiveness associated with each respective media content for controlling (e.g., changing, maintaining) a value of the at least one biometric parameter. For example, each respective media content of the set of media content may be scored based on a respective effectiveness associated with each respective media content for controlling (e.g., changing, maintaining) a value of the heart rate, the respiratory rate, or the like. In some aspects, each respective media content of the set of media content may be scored based on a pattern (e.g., a model, historical data) associated with a respective effectiveness associated with each respective media content for controlling a value of the heart rate, the respiratory rate, or the like. In some other aspects, each respective media content of the set of media content may be scored based on instantaneous biometric feedback (e.g., a user’s heart rate drops) while the user’s is experiencing the media content (e.g., listening, watching, etc.). In some other aspects, each respective media content of the set of media content may be scored over a time interval (e.g., sleep latency gradually decreasing over a couple of weeks, a user’s REM sleep gradually increasing, etc.). The scores may be based on how the media content affects a particular user (e.g., the scores may differ between users), or the scores may be based on how the media content generally affects a population of physiologically or socially similar users (e.g., athletes, senior citizens, users within particular age ranges or genders, etc.)

In some implementations, any of the components of the system 100, including the ring 104-a, the user device 106-a associated with User 1, the one or more servers 110, or any combination thereof, may generate recommendations for User 1 (e.g., via the ring 104-a, the user device 106-a, or both) based on the selected media content. In some other implementations, any of the components of the system 100, including the ring 104-a, the user device 106-a associated with User 1, the one or more servers 110, or any combination thereof, may generate alerts or messages for User 1 (e.g., via the ring 104-a, the user device 106-a, or both) based on the selected media content, where the alerts or messages may provide insights regarding biometrics of the User 1. For example, the alerts or messages may provide insights regarding changes to biometrics, such as a heart rate, a respiratory rate, or the like of the User 1, based on an involvement from the User 1 with the selected media content recommended to the User 1 (e.g., listening, reading, watching, or the like).

It should be appreciated by a person skilled in the art that one or more aspects of the disclosure may be implemented in the system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

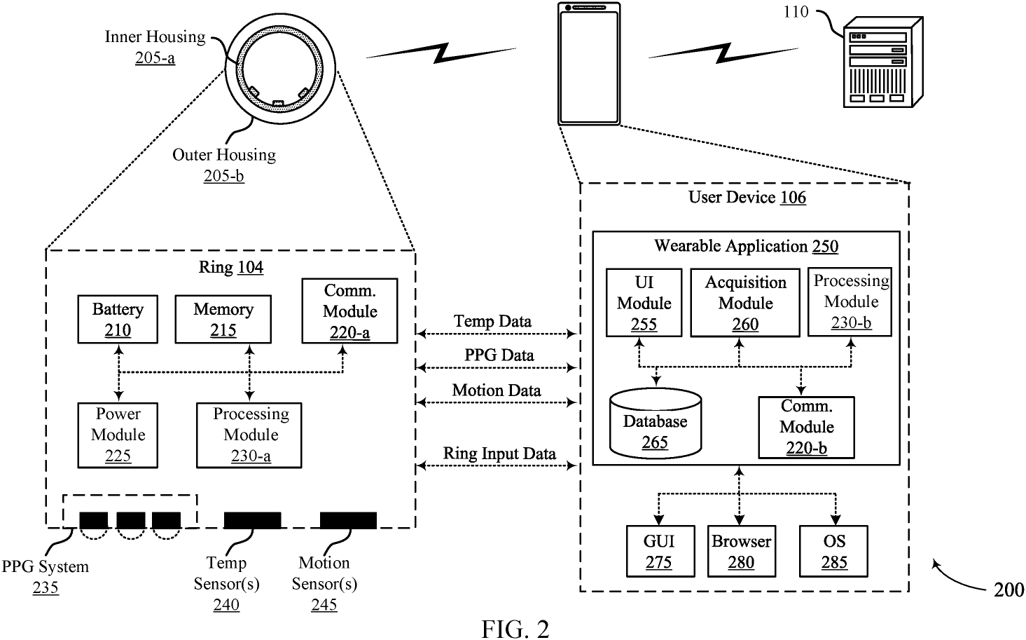

FIG. 2 illustrates an example of a system 200 that supports content delivery techniques for controlling biometric parameters in accordance with aspects of the present disclosure. The system 200 may implement, or be implemented by, system 100. In particular, the system 200 illustrates an example of a ring 104 (e.g., wearable device 104), a user device 106, and a server 110, as described with reference to FIG. 1.

In some examples, the ring 104 may be configured to be worn around a user’s finger, and may determine one or more user physiological parameters when worn around the user’s finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some examples, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or another sensor) may be fabricated. In another specific example, a temperature sensor 240 (or other sensor) may be attached to a user’s finger (e.g., using a clamp, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user’s finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user’s finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some examples, a charger or other power source may include additional sensors that may be used to collect data in addition to, or supplemented, data collected by the ring 104 itself. Moreover, a charger or other power source for the ring 104 may function as a user device 106, and the charger or other power source for the ring 104 may be configured to receive data from the ring 104, store and/or process data received from the ring 104, and communicate data between the ring 104 and the servers 110.

In some examples, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user’s finger (e.g., skin temperature). In some implementations, the temperature sensor 240 may contact the user’s skin. In other implementations, a portion of the housing 205 (e.g., the inner housing 205-a) may form a barrier (e.g., a thin, thermally conductive barrier) between the temperature sensor 240 and the user’s skin. In some implementations, portions of the ring 104 configured to contact the user’s finger may have thermally conductive portions and thermally insulative portions. The thermally conductive portions may conduct heat from the user’s finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user’s temperature over time. For example, the processing module 230-a may sample the user’s temperature according to a sampling rate. An example sampling rate may include one sample per second, although the processing module 230-a may be configured to sample the temperature signal at other sampling rates that are higher or lower than one sample per second. In some implementations, the processing module 230-a may sample the user’s temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate may be stored in memory 215 and may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user’s finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user’s finger (e.g., any finger). For example, one or more temperature sensors 240 on the ring 104 may acquire a user’s temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user’s finger may differ from the temperature measured at a user’s wrist or other external body location. Additionally, the distal temperature measured at a user’s finger (e.g., a “shell” temperature) may differ from the user’s core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user’s finger. The PPG signal generated by the PPG system 235 may indicate the perfusion of blood in the illuminated region. For example, the PPG signal may indicate blood volume changes in the illuminated region caused by a user’s pulse pressure. The processing module 230-a may sample the PPG signal and determine a user’s pulse waveform based on the PPG signal. The processing module 230-a may determine a variety of physiological parameters based on the user’s pulse waveform, such as a user’s respiratory rate, heart rate, HRV, oxygen saturation, and other circulatory parameters.

In some implementations, the PPG system 235 may be configured as a reflective PPG system 235 including the optical receiver(s) to receive transmitted light that is reflected through the region of the user’s finger. In some implementations, the PPG system 235 may be configured as a transmissive PPG system 235 including the optical transmitter(s) and optical receiver(s) that may be arranged opposite to one another, such that light is transmitted directly through a portion of the user’s finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user’s heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user’s respiratory rate over time. For example, the processing module 230-a may determine respiratory rate based on frequency modulation, amplitude modulation, or baseline modulation of the user’s IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a sleep score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user’s finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user’s physiological parameters may be measured by sensors included on a ring 104, other devices may measure a user’s physiological parameters. For example, although a user’s temperature may be measured by a temperature sensor 240 included in a ring 104, other devices may measure a user’s temperature. In some examples, other wearable devices (e.g., wrist devices) may include sensors that measure user physiological parameters. Additionally, medical devices, such as external medical devices (e.g., wearable medical devices) and/or implantable medical devices, may measure a user’s physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some examples, the user device 106 includes a wearable application 250, an operating system (OS) 285, a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some examples, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user’s sleep for a given “sleep day.” In some examples, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time in that the respective users typically sleep.

In some implementations, each overall score for a user for each respective day (e.g., Sleep Score, Readiness Score) may be determined/calculated based on one or more “contributors,” “factors,” or “contributing factors.” For example, a user’s overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user’s sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user’s overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user’s needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user’s sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user’s resting heart rate to stabilize during the night. A sign of a very good recovery is that the user’s resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user’s highest temperature during the nap is at least 0.5° C. higher than the highest temperature during the longest period. In some examples, the ring may measure a user’s body temperature while the user is asleep, and the system 200 may display the user’s average temperature relative to the user’s baseline temperature. If a user’s body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for content delivery for controlling biometrics. In particular, the system 200 illustrated in FIG. 2 may support techniques for recommending media content to a user by causing the user device 106 corresponding to the user to display recommended media content. For example, as shown in FIG. 2, a user may be associated with the ring 104 and the user device 106. In this example, the ring 104 may collect biometric data associated with the user, including heart rate, respiratory rate, skin temperature, and the like. In some aspects, biometric data collected by the ring 104 may be used to select the media content (e.g., music, audio book, podcast, video, etc.) for recommending to the user. The selection of the media content may be performed by any of the components of the system 200. Upon selecting the media content (e.g., music, audio book, podcast, video, etc.) for recommending to the user, the system 200 may selectively cause the GUI 275 of the user device 106 to display an indication of the recommended media content.

As noted previously herein, the ring 104 of the system 200 may be worn by a user to collect biometric data from the user, including temperature, heart rate, respiration rate, and the like. The ring 104 of the system 200 may collect the biometric data from the user based on arterial blood flow. The biometric data may be collected continuously. In some implementations, the biometric data may be collected periodically or aperiodically. In some implementations, the acquisition module 260 may receive the collected biometric data from the ring 104 and forward the biometric data to the processing module 230-b that may determine that at least one biometric parameter associated with the biometric data satisfies a threshold during an occasion. For example, upon receiving the biometric data associated with the user from the ring 104, the processing module 230-b may determine that at least one biometric parameter, such as a heart rate, a respiratory rate, or the like satisfies a threshold during a time interval (e.g., the resting heart rate has exceeded a preconfigured value or has deviated from that user’s baseline for more than 10 minutes). Any of the components of the user device 106 may then select media content from a set of media content for recommending to the user.

In some cases, any of the components of the system 200 may score each respective media content of the set of media content (e.g., music, audio book, podcast, video, etc.) based on a respective effectiveness associated with each respective media content for controlling (e.g., changing, maintaining) a value of the at least one biometric parameter. For example, each respective media content of the set of media content may be scored based on a respective effectiveness associated with each respective media content for controlling a value of the heart rate, the respiratory rate, or the like. In some implementations, any of the components of the system 200 may generate recommendations for the user (e.g., via the ring 104, the user device 106, or both) based on the selected media content. In some other implementations, any of the components of the system 200 may generate alerts or messages for the user (e.g., via the ring 104, the user device 106, or both) based on the selected media content, where the alerts or messages may provide insights regarding biometrics of the user as described herein.

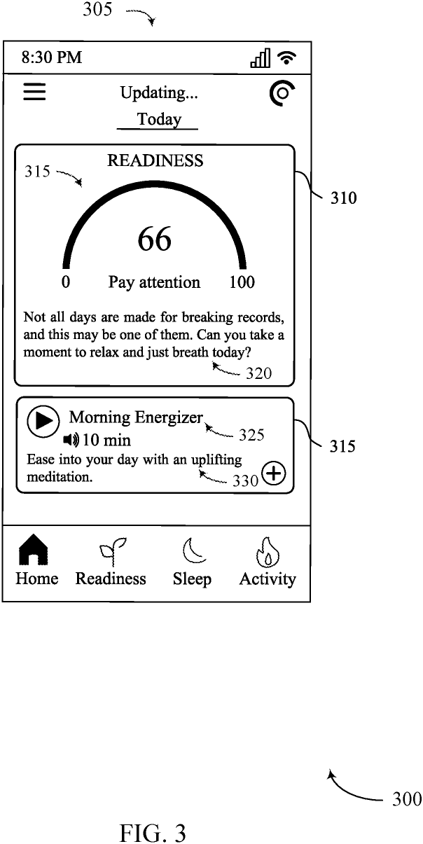

FIG. 3 illustrates an example of a GUI 300 that supports content delivery techniques for controlling biometric parameters in accordance with aspects of the present disclosure. The GUI 300 may implement, or be implemented by, aspects of the system 100 or the system 200, or any combination thereof. In some examples, the GUI 300 may be an example of a GUI of a user device that may be examples of GUIs and user devices as described with reference to FIGS. 1 and 2. For example, the GUI 300 may be an example of a GUI 275 of a user device 106 as described with reference to FIGS. 1 and 2. In the example of FIG. 3, the GUI 300 may include an application interface 305 that may be displayed to a user 102 via the GUI 300.

The application interface 305 may be associated with an application running on a user device 106. In some examples, the application interface 305 may include a set of graphical elements (also referred to as widgets or components) the application provides so that a user 102 may provide input to, and receive output from, the application via the application interface 305. In some examples, one or more operations associated with the GUI 300 may be performed based on a manipulation of the one or more graphical elements associated with the GUI 300. Examples of graphical elements associated with the GUI 300 may include, but are not limited to, buttons, sliders, droplists, tabs, text boxes, and the like. The application interface 305 may also include a set of tabs enabling the user 102 to switch between different features of the application. For example, the set of tabs may allow the user 102 to switch between one or more of a “home feature,” a “readiness feature,” a “sleep feature,” or an “activity feature” in the application running on the user device 106.

As noted previously, biometric data collected from the user 102 may be used to calculate a score and/or a metric (e.g., a Health Risk Score, a Sleep Score, a Readiness Score) for the user 102. The calculated score and/or metric may be displayed to the user 102 via the GUI 300, as shown in the application interface 305. For example, the application interface 305 may include a graphical element 310 that may display one or more of a Readiness Score 315 or a message 320. In some examples, the message 320 may be a configurable message associated with a potential health risk for the user 102. For example, the message 320 may be configurable (e.g., customizable), such that the user 102 may receive different messages based on different Health Risk Scores, as described herein.

In the example of FIG. 3, the application interface 305 may include a graphical element 315 that may display a media content recommendation to a user 102. For example, the graphical element 315 may include a media content 325 and media content information 330. A user 102 may be provided the media content recommendation based on biometric data associated with the user 102. For example, a user 102 may be associated with a wearable device 104 (e.g., ring 104) and a user device 106. In this example, the ring 104 may collect biometric data associated with the user 102, including heart rate, respiratory rate, and the like. In some aspects, biometric data collected by the ring 104 may be used to select the media content 325 (e.g., music, audio book, podcast, video, etc.) for recommending to the user 102. For example, the biometric data may serve as a trigger for recommending media content 325 to the user 102.

The selection of the media content 325 may be performed by any of the components of the system 100 and/or the system 200, including a ring 104, a user device 106 associated with a user 102, the one or more servers 110, or any combination thereof. Upon selecting the media content 325 (e.g., music, audio book, podcast, etc.) for recommending to the user 102, the GUI 300 corresponding to the user device 106 may display a representation of the recommended media content 325, for example, via the graphical element 315. Upon receiving the biometric data, any of the components of the system 100 and/or the system 200 may determine that at least one biometric parameter associated with the biometric data satisfies a threshold during an occasion. For example, upon receiving the biometric data associated with the user 102 from the ring 104, any of the components of the system 100 and/or the system 200, including the ring 104, the user device 106 associated with the user 102, the one or more servers 110, or any combination thereof, may determine that at least one biometric parameter, such as a heart rate, a respiratory rate, or the like satisfies a threshold during a time interval. The threshold may be specific to the specific user 102 (e.g., based on their historical or baseline values for a physiological or biometric parameter) or may be configured for all users or for particular groups of users (e.g., based on age, gender, activity level, etc.).

The media content 325 may be associated with a score based on a respective effectiveness associated with the media content 325 for controlling (e.g., changing, adjusting, maintaining) a value of the at least one biometric parameter. For example, the media content 325 may be scored based on a respective effectiveness for changing or maintaining a value of the heart rate, the respiratory rate, or the like, for the user 102. Any of the components of the system 100 and/or the system 200 may generate baseline biometric data for the user 102 (e.g., customized baseline ranges) based on acquired biometric data. The acquired biometric data may be used to generate baseline temperature data, baseline respiratory rate data, baseline HRV data, and the like. As such, the score may indicate the effectiveness for controlling the value of the at least one biometric parameter respective to its baseline value. Any of the components of the system 100 and/or the system 200, including the ring 104, the user device 106 associated with the user 102, the one or more servers 110, or any combination thereof, may select media content 325 from a set of media content for recommending to the user 102 via the GUI 300.

In some implementations, the selection of the media content 325 may be based on a crowdsourcing-based media content recommendation. Any of the components of the system 100 and/or the system 200, including a ring 104, a user device 106 associated with a user 102, the one or more servers 110, or any combination thereof, may correlate at least one biometric parameter to a set of users 102 associated with the at least one biometric parameter and the media content 325. One or more users of the set of users 102 may have previously been recommended the media content 325. For example, any of the components of the system 100 and/or the system 200, including a ring 104, a user device 106 associated with a user 102, the one or more servers 110, or any combination thereof, may determine a group of users 102 that may have had a similar (e.g., within a range) or the same heart rate, respiratory rate, or the like, as the user 102. Additionally or alternatively, any of the components of the system 100 and/or the system 200, including a ring 104, a user device 106 associated with a user 102, the one or more servers 110, or any combination thereof, may determine or select a group of users 102 based on similar biometric parameters (e.g., same elevated heart rate value), and/or that the group of users 102 is determined or selected based at least in part on similar social and/or physiological parameters (age, level of fitness, gender, pregnancy status, etc.). Based on this determination, any of the components of the system 100 and/or the system 200, including a ring 104, a user device 106 associated with a user 102, the one or more servers 110, or any combination thereof, may identify media content that have been previously recommended to the group of users 102. For example, any of the components of the system 100 and/or the system 200, including a ring 104, a user device 106 associated with a user 102, the one or more servers 110, or any combination thereof, may identify that the media content 325 has been previously recommended to the group of users 102. As a result, the media content 325 may be selected and displayed to the user 102 via the GUI 300.

In some other implementations, the selection of the media content 325 may be based on previous biometric data collected from the ring 104. Any of the components of the system 100 and/or the system 200, including a ring 104, a user device 106 associated with a user 102, the one or more servers 110, or any combination thereof, may determine that at least one biometric parameter satisfied the threshold during a previous occasion, and determine that the media content was previously selected for recommending to the user 102 during the previous occasion. For example, any of the components of the system 100 and/or the system 200, including a ring 104, a user device 106 associated with a user 102, the one or more servers 110, or any combination thereof, may determine that a heart rate, a respiratory rate, or the like associated with the user 102 satisfied the threshold during a previous time interval. Any of the components of the system 100 and/or the system 200, including a ring 104, a user device 106 associated with a user 102, the one or more servers 110, or any combination thereof, may then determine that the media content 325 was previously selected for recommending to the user 102. As a result, the media content 325 may be displayed to the user 102 via the GUI 300.

The selection of the media content 325 may be based on a profile associated with the user 102. Any of the components of the system 100 and/or the system 200, including a ring 104, a user device 106 associated with a user 102, the one or more servers 110, or any combination thereof, may analyze a profile associated with the user 102 based at least in part on at least one biometric parameter satisfying the threshold. For example, any of the components of the system 100 and/or the system 200, including a ring 104, a user device 106 associated with a user 102, the one or more servers 110, or any combination thereof, may determine that a heart rate, a respiratory rate, or the like associated with the user 102 satisfied the threshold, and analyze a profile associated with the user 102 to determine the media content 325 for recommending to the user 102.

The profile may be stored in memory (e.g., the database 265 as described with reference to FIG. 2) of the user device 106 corresponding to the user 102. In some examples, the profile associated with the user 102 may indicate one or more media content associated with a set of media content previously recommended to the user 102. Additionally or alternatively, the profile may indicate a user feedback associated with the one or more media content associated with the set of media content previously recommended to the user 102. For example, the user feedback may indicate whether the user 102 “likes” or “dislikes” the previously recommended media content. As such, any of the components of the system 100 and/or the system 200, including a ring 104, a user device 106 associated with a user 102, the one or more servers 110, or any combination thereof, may determine whether to recommend or not recommend certain media content by applying one or more filters to media content based on the user feedback. In some examples, the user feedback may include a ranking or a scoring by the user 102 of media content previously or currently recommended media content to the user 102. For example, the user 102 inputs manually that they found certain media content to be helpful/effective. In addition, the system may collect biometric feedback from the user 102 before, during, or after the user 102 interacts with a piece of media content (e.g., in the form of measured heart rate, respiration rate, skin temperature, or aggregate scores such as Sleep Scores or Readiness Scores). This biometric feedback may be used by the system, alone or in addition to other inputs or feedback, to score media content and/or recommend media content to the user 102 in the future. Additionally or alternatively, media content may be recommended to the user 102 based on user feedback from other users. For example, media content may be recommended to the user 102 based on media content other users liked or disliked.

In some other implementations, the selection of the media content 325 may be based on an activity of the user 102. Any of the components of the system 100 and/or the system 200, including a ring 104, a user device 106 associated with a user 102, the one or more servers 110, or any combination thereof, may determine an activity of the user 102 based at least in part on biometric data collected and received from the ring 104, and based on the determined activity, any of the components of the system 100 and/or the system 200, including a ring 104, a user device 106 associated with a user 102, the one or more servers 110, or any combination thereof may select the media content 325 for recommending to the user 102. In some examples, any of the components of the system 100 and/or the system 200, including a ring 104, a user device 106 associated with a user 102, the one or more servers 110, or any combination thereof may correlate the activity associated with the user 102 to one or more of the biometric parameter (e.g., heart rate, or the like) associated with the user 102 and the media content 325. The activity may include a physical activity (e.g., a fitness activity) that the user is in a non-resting state or a nonphysical activity that the user 102 is in a resting state.

Any of the components of the system 100 and/or the system 200, including a ring 104, a user device 106 associated with a user 102, the one or more servers 110, or any combination thereof, may determine multiple media content based at least in part on the collected biometric data associated with the user 102. The selection of the media content 325 may be based on a respective score associated with each respective media content of the various media content. The respective score may indicate the effectiveness associated with each respective media content for controlling the value of the at least one biometric parameter (e.g., heart rate). The scores associated with the media content may change over time based on feedback received from a user 102. For example, the system may detect that the heart rate of the user 102 drops most significantly after listening to a particular guided meditation, and may therefore increase the score associated with that meditation that may result in that meditation being more likely to be recommended in the future (e.g., in response to the system recognizing that the user’s heart rate is elevated and/or in response to the user indicating a desire for a meditation that is effective at lowering heart rate).

Therefore, the GUI 300 may provide personalized media content recommendations to the user 102 in real-time based at least in part on biometric data associated with the user 102. By providing personalized media content recommendations to the user 102 in real-time triggered by biometric data associated with the user 102, the user 102 may experience an improvement to one or more biometric parameters (e.g., heart rate, respiratory rate, and the like) associated with the biometric data.

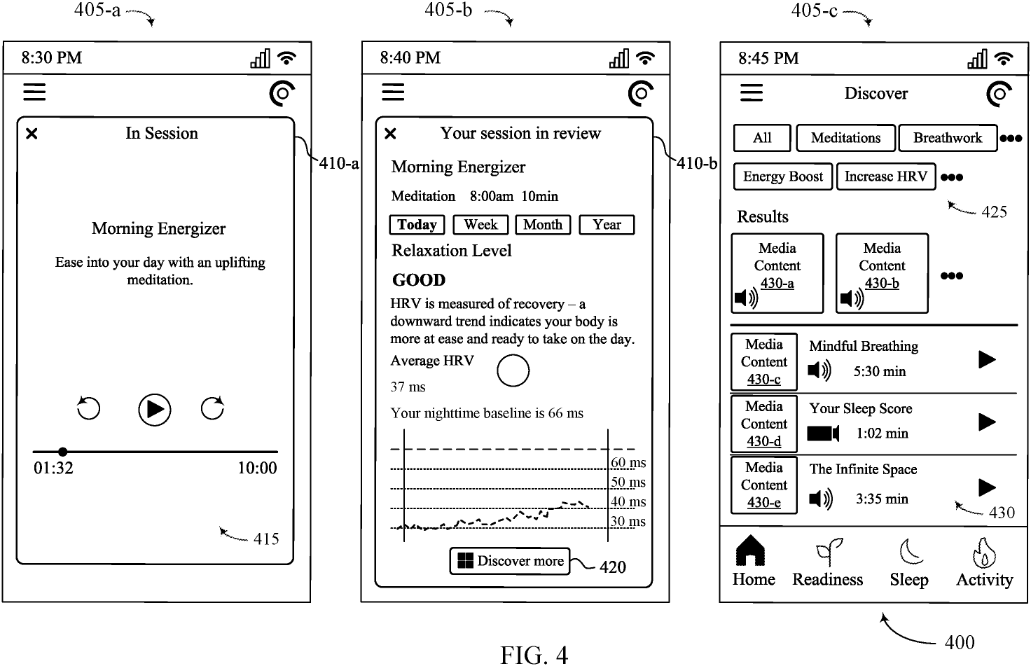

FIG. 4 illustrates an example of a GUI 400 that supports content delivery techniques for controlling biometric parameters in accordance with aspects of the present disclosure. The GUI 400 may implement, or be implemented by, aspects of the system 100 or the system 200, or any combination thereof. In some examples, the GUI 400 may be an example of a GUI of a user device that may be examples of GUIs and user devices as described with reference to FIGS. 1 and 2. For example, the GUI 400 may be an example of a GUI 275 of a user device 106 as described with reference to FIGS. 1 and 2. In the example of FIG. 4, the GUI 400 may include a sequence of applications interfaces including one or more of an application interface 405-a, an application interface 405-b, or an application interface 405-c that may be displayed to a user 102 via the GUI 400.

The application interface 405-a may be associated with an application running on a user device 106. In some examples, the application interface 405-a may include a set of graphical elements the application provides so that a user 102 may provide input to, and receive output from, the application via the application interface 405-a. For example, the application interface 405-a may include a graphical element 410-a that may display recommended media content 415 (e.g., audio content, video content, or the like) to a user 102 corresponding to a user device 106 associated with the GUI 400. As described herein, the selection of the media content 415 may be performed by any of the components of the system 100 and/or the system 200, including a ring 104, a user device 106 associated with a user 102, the one or more servers 110, or any combination thereof, and may be based at least in part on biometric data collected by the ring 104.

In some implementations, the application interface 405-a may include one or more graphical elements associated with the media content 415 including, but not limited to, buttons, sliders, droplists, tabs, text boxes, and the like. For example, the application interface 405-a may receive an input from the user 102 via one or more graphical elements associated with the GUI 400 to enable the media content 415 for a duration associated with the media content 415. In other words, the application interface 405-a may receive an input from the user 102 to play the media content 415. In some implementations, any of the components of the system 100 and/or the system 200, including a ring 104, a user device 106 associated with a user 102, the one or more servers 110, or any combination thereof, may monitor for biometric feedback data associated with the user 102 (e.g., from the ring 104) during the duration (e.g., the entire duration or a portion of the duration) associated with the media content 415.

Any of the components of the system 100 and/or the system 200, including a ring 104, a user device 106 associated with a user 102, the one or more servers 110, or any combination thereof, may receive the biometric feedback data associated with the user 102 (e.g., from the ring 104) during the duration associated with the media content 415 and determine a biometric change to the at least one biometric parameter associated with the biometric data based on the biometric feedback data associated with the user 102. For example, any of the components of the system 100 and/or the system 200, including a ring 104, a user device 106 associated with a user 102, the one or more servers 110, or any combination thereof, may determine a change to a heart rate, a respiratory rate, or the like associated with the user 102. Any of the components of the system 100 and/or the system 200, including a ring 104, a user device 106 associated with a user 102, the one or more servers 110, or any combination thereof, may output a representation of the biometric change via the GUI 400.

In the example of FIG. 4, the application interface 405-b may include a graphical element 410-b that may display the biometric change corresponding to a biometric parameter (e.g., heart rate, or the like) associated with the user 102. The graphical element 410-b may also provide additional information associated with the user 102. For example, the graphical element 410-b may display a summary report to the user 102 including the biometric change corresponding to a biometric parameter (e.g., heart rate, or the like) associated with the user 102 along with a message that may be configurable. In some implementations, the application interface 405-b may display a graphical element 420 (e.g., “Discover more”) that may enable the user 102 to browse additional media content.

Upon receiving an input from the user 102 selecting the graphical element 420, the GUI 400 may display the application interface 405-c. The application interface 405-c may display a set of criteria 425 and a set of media content 430 (e.g., a media content 430-a, a media content 430-b, a media content 430-c, a media content 430-d, and a media content 430-e) associated with the set of criteria 425. The set of criteria 425 may be used by any of the components of the system 100 and/or the system 200, including a ring 104, a user device 106 associated with a user 102, the one or more servers 110, or any combination thereof to filter (e.g., rank, order, list) the set of media content 430. Examples of media content may include audio content or video content. In some implementations, each media content 430 may include an icon indicating whether the media content is an audio content or a video content.

A criterion associated with the set of criteria may correspond to a biometric parameter (e.g., HRV or the like). Examples of criterion of the set of criteria may include, but are not limited to, “mediations,” “breathwork,” “energy boost,” “increased HRV,” or the like. In some implementations, any of the components of the system 100 and/or the system 200, including a ring 104, a user device 106 associated with a user 102, the one or more servers 110, or any combination thereof may display the set of media content 430 based at least in part on, for example, an effectiveness associated with each media content 430 for changing or maintaining a value of at least one biometric parameter as described herein. For example, the application interface 405-c may display the set of media content 430 based at least in part on the ranking.

Therefore, the GUI 400 may provide personalized media content recommendations to the user 102 in real-time based at least in part on biometric data associated with the user 102. By providing personalized media content recommendations to the user 102 in real-time triggered by biometric data associated with the user 102, the user 102 may experience an improvement to one or more biometric parameters (e.g., heart rate, respiratory rate, and the like) associated with the biometric data.

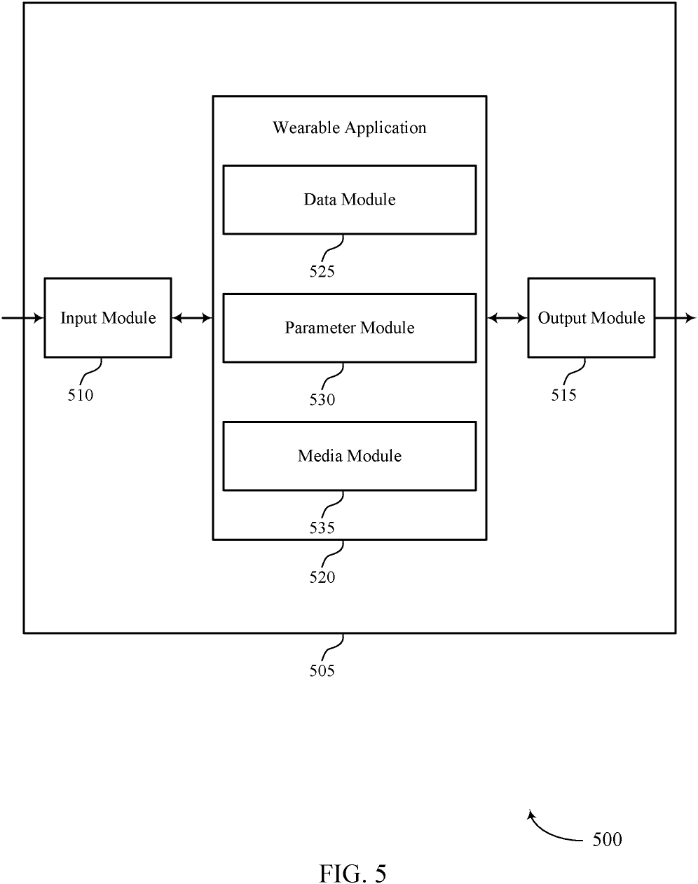

FIG. 5 shows a block diagram 500 of a device 505 that supports content delivery techniques for controlling biometric parameters in accordance with aspects of the present disclosure. The device 505 may include an input module 510, an output module 515, and a wearable application 520. The device 505 may also include a processor. Each of these components may be in communication with one another (e.g., via one or more buses).

The input module 510 may provide a means for receiving information such as packets, user data, control information, or any combination thereof associated with various information channels (e.g., control channels, data channels, information channels related to content delivery techniques for controlling biometric parameters). Information may be passed on to other components of the device 505. The input module 510 may utilize a single antenna or a set of multiple antennas.

The output module 515 may provide a means for transmitting signals generated by other components of the device 505. For example, the output module 515 may transmit information such as packets, user data, control information, or any combination thereof associated with various information channels (e.g., control channels, data channels, information channels related to content delivery techniques for controlling biometric parameters). In some examples, the output module 515 may be co-located with the input module 510 in a transceiver module. The output module 515 may utilize a single antenna or a set of multiple antennas.

For example, the wearable application 520 may include a data module 525, a parameter module 530, a media module 535, or any combination thereof. In some examples, the wearable application 520, or various modules thereof, may be configured to perform various operations (e.g., receiving, monitoring, transmitting) using or otherwise in cooperation with the input module 510, the output module 515, or both. For example, the wearable application 520 may receive information from the input module 510, send information to the output module 515, or be integrated in combination with the input module 510, the output module 515, or both to receive information, transmit information, or perform various other operations as described herein.

The wearable application 520 may support content delivery at the device 505 in accordance with examples as disclosed herein. The data module 525 may be configured as or otherwise support a means for receiving biometric data associated with a user from a wearable device (e.g., a ring 104). The parameter module 530 may be configured as or otherwise support a means for determining that at least one biometric parameter of a set of biometric parameters associated with the biometric data satisfies a threshold during an occasion. The media module 535 may be configured as or otherwise support a means for selecting media content from a set of media content for recommending to the user. Each respective media content of the set of media content may be scored based at least in part on a respective effectiveness associated with each respective media content for controlling a value of the at least one biometric parameter. In some examples, the selecting may be triggered based at least in part on the at least one biometric parameter satisfying the threshold. In some examples, the media content may be selected based at least in part on a score associated with the media content. The media module 535 may be configured as or otherwise support a means for outputting the media content via a GUI of the device 505 during the occasion.

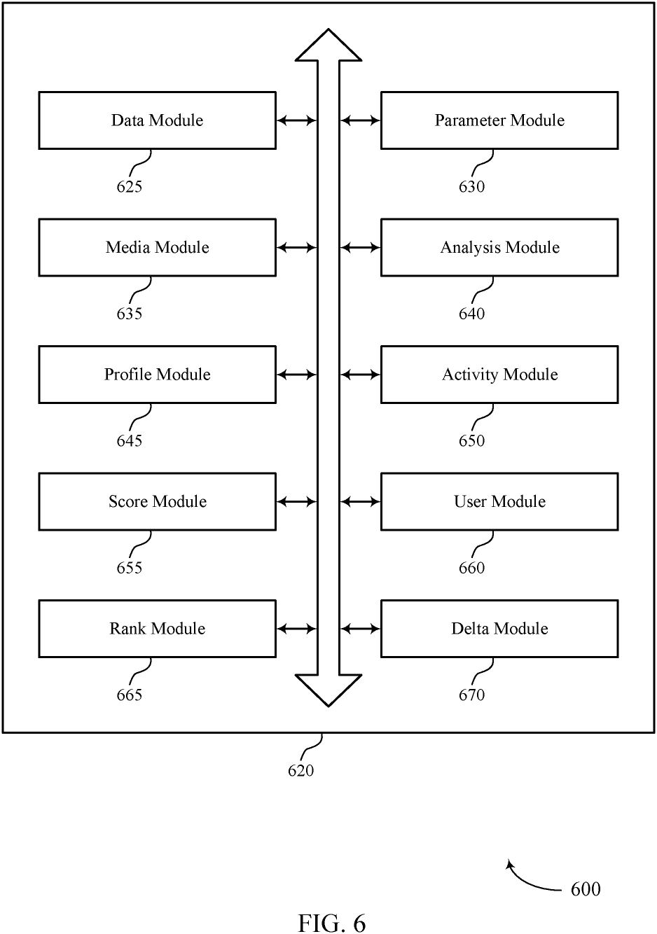

FIG. 6 shows a block diagram 600 of a wearable application 620 that supports content delivery techniques for controlling biometric parameters in accordance with aspects of the present disclosure. The wearable application 620 may be an example of aspects of a wearable application or a wearable application 520, or both, as described herein. The wearable application 620, or various modules thereof, may be an example of means for performing various aspects of content delivery techniques for controlling biometric parameters as described herein. For example, the wearable application 620 may include a data module 625, a parameter module 630, a media module 635, an analysis module 640, a profile module 645, an activity module 650, a score module 655, a user module 660, a rank module 665, a delta module 670, or any combination thereof. Each of these modules may communicate, directly or indirectly, with one another (e.g., via one or more buses).

The wearable application 620 may support content delivery at a device in accordance with examples as disclosed herein. The data module 625 may be configured as or otherwise support a means for receiving biometric data associated with a user from a wearable device. The parameter module 630 may be configured as or otherwise support a means for determining that at least one biometric parameter of a set of biometric parameters associated with the biometric data satisfies a threshold during an occasion. The media module 635 may be configured as or otherwise support a means for selecting media content from a set of media content for recommending to the user. Each respective media content of the set of media content may be scored based at least in part on a respective effectiveness associated with each respective media content for controlling a value of the at least one biometric parameter. In some examples, the selecting may be triggered based at least in part on the at least one biometric parameter satisfying the threshold. In some examples, the media content may be selected based at least in part on a score associated with the media content. In some examples, the media module 635 may be configured as or otherwise support a means for outputting the media content via a GUI of the device during the occasion.

The analysis module 640 may be configured as or otherwise support a means for correlating the at least one biometric parameter to a set of users associated with the at least one biometric parameter and the media content, one or more users of the set of users previously being recommended the media content. In some examples, the media module 635 may be configured as or otherwise support a means for selecting the media content based at least in part on correlating the at least one biometric parameter to the set of users associated with the at least one biometric parameter and the media content.

In some examples, the parameter module 630 may be configured as or otherwise support a means for determining that the at least one biometric parameter satisfied the threshold during a previous occasion. In some examples, the media module 635 may be configured as or otherwise support a means for determining that the media content was previously selected for recommending to the user during the previous occasion. In some examples, the media module 635 may be configured as or otherwise support a means for selecting the media content based at least in part on determining that the at least one biometric parameter satisfied the threshold during the previous occasion and the media content was previously selected for recommending to the user during the previous occasion.

The profile module 645 may be configured as or otherwise support a means for analyzing a profile associated with the user based at least in part on the at least one biometric parameter satisfying the threshold. In some examples, the profile may indicate one or more media content associated with the set of media content previously recommended to the user. In some examples, the profile may indicate a user feedback associated with the one or more media content associated with the set of media content previously recommended to the user. In some examples, the media module 635 may be configured as or otherwise support a means for selecting the media content based at least in part on analyzing the profile associated with the user.

In some examples, the activity module 650 may be configured as or otherwise support a means for determining an activity associated with the user during the occasion. In some examples, the analysis module 640 may be configured as or otherwise support a means for correlating the activity associated with the user to one or more of the at least one biometric parameter and the media content. In some examples, the media module 635 may be configured as or otherwise support a means for selecting the media content based at least in part on the correlating the activity associated with the user to one or more of the at least one biometric parameter and the media content.

The score module 655 may be configured as or otherwise support a means for identifying a respective score associated with each respective media content of the set of media content. In some examples, the respective score may indicate the respective effectiveness associated with each respective media content of the set of media content for controlling the value of the at least one biometric parameter. In some examples, the media module 635 may be configured as or otherwise support a means for selecting the media content based at least in part on identifying the respective score associated each respective media content of the set of media content.

In some examples, the user module 660 may be configured as or otherwise support a means for receiving an input from the user via the GUI of the device. In some examples, the media module 635 may be configured as or otherwise support a means for enabling the media content for a duration associated with the media content based at least in part on receiving the input from the user via the GUI of the device. In some examples, the data module 625 may be configured as or otherwise support a means for monitoring for biometric feedback data associated with the user from the wearable device during the duration associated with the media content based at least in part on the enabling.

The data module 625 may be configured as or otherwise support a means for receiving the biometric feedback data associated with the user from the wearable device during the duration associated with the media content based at least in part on the monitoring. In some examples, the delta module 670 may be configured as or otherwise support a means for determining a biometric change to the at least one biometric parameter of the set of biometric parameters associated with the biometric data based at least in part on the biometric feedback data associated with the user. In some examples, the delta module 670 may be configured as or otherwise support a means for outputting a representation of the biometric change to the biometric data via the GUI of the device.

In some examples, the rank module 665 may be configured as or otherwise support a means for assigning a rank to the media content for recommending to the user during a subsequent occasion and for ordering the media content in a list of media content provided to the user via the GUI of the device based at least in part on the biometric change to the at least one biometric parameter of the set of biometric parameters associated with the biometric data.

In some examples, the media content comprises audio content or video content. In some examples, the at least one biometric parameter of the set of biometric parameters associated with the biometric data comprises heart rate data associated with the user, respiratory rate data associated with the user, sleep data associated with the user, activity data associated with the user, or any combination thereof. In some examples, the wearable device comprises a wearable ring device.

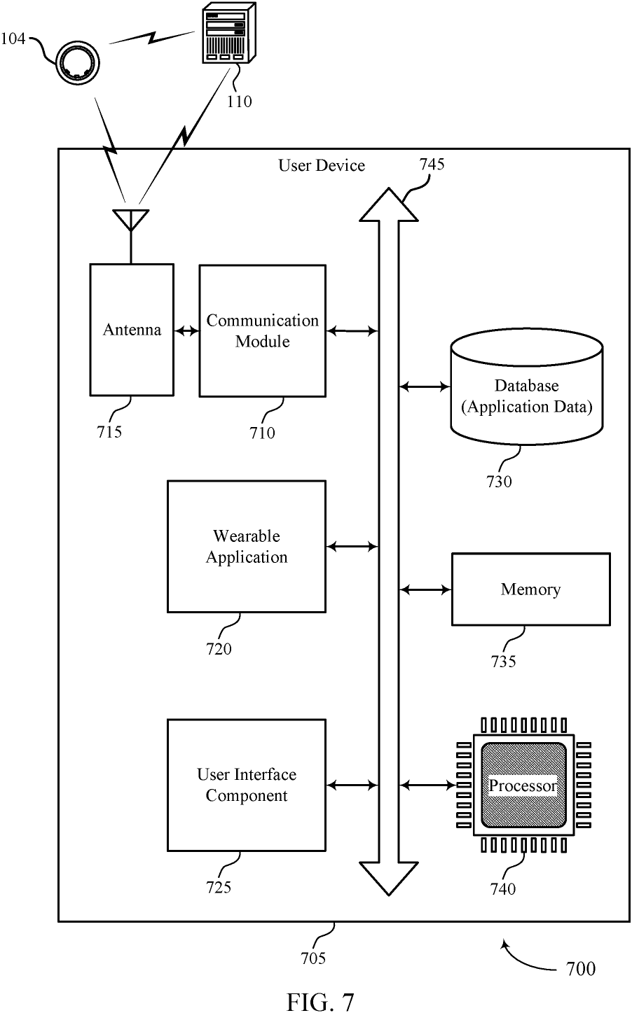

FIG. 7 shows a diagram of a system 700 including a device 705 that supports content delivery techniques for controlling biometric parameters in accordance with aspects of the present disclosure. The device 705 may be an example of or include the modules of a device 505 as described herein. The device 705 may include modules for bi-directional voice and data communications including components for transmitting and receiving communications, such as a wearable application 720, a communication module 710, an antenna 715, a user interface component 725, a database (application data) 730, a memory 735, and a processor 740. These components may be in electronic communication or otherwise coupled (e.g., operatively, communicatively, functionally, electronically, electrically) via one or more buses (e.g., a bus 745).

The communication module 710 may manage input and output signals for the device 705 via the antenna 715. The communication module 710 may include an example of the communication module 220-b of the user device 106 shown and described in FIG. 2. For example, the communication module 710 may manage communications with the ring 104 and the server 110, as illustrated in FIG. 2. The communication module 710 may also manage peripherals not integrated into the device 705. In some cases, the communication module 710 may represent a physical connection or port to an external peripheral. In some cases, the communication module 710 may utilize an operating system such as iOS®, ANDROID®, MS-DOS®, MS-WINDOWS®, OS/2®, UNIX®, LINUX®, or another known operating system. In other cases, the communication module 710 may represent or interact with a wearable device (e.g., ring 104), modem, a keyboard, a mouse, a touchscreen, or a similar device. In some cases, the communication module 710 may be implemented as part of the processor 740. In some examples, a user may interact with the device 705 via the communication module 710, user interface component 725, or via hardware components controlled by the communication module 710.

In some cases, the device 705 may include a single antenna 715. However, in some other cases, the device 705 may have more than one antenna 715 that may be capable of concurrently transmitting or receiving multiple wireless transmissions. The communication module 710 may communicate bi-directionally, via the one or more antennas 715, wired, or wireless links as described herein. For example, the communication module 710 may represent a wireless transceiver and may communicate bi-directionally with another wireless transceiver. The communication module 710 may also include a modem to modulate packets, to provide the modulated packets to one or more antennas 715 for transmission, and to demodulate packets received from the one or more antennas 715.

The user interface component 725 may manage data storage and processing in a database 730. In some cases, a user may interact with the user interface component 725. In other cases, the user interface component 725 may operate automatically without user interaction. The database 730 may be an example of a single database, a distributed database, multiple distributed databases, a data store, a data lake, or an emergency backup database.

The memory 735 may include RAM and ROM. The memory 735 may store computer-readable, computer-executable software including instructions that, when executed, cause the processor 740 to perform various functions described herein. In some cases, the memory 735 may contain, among other things, a BIOS that may control basic hardware or software operation such as the interaction with peripheral components or devices.

The processor 740 may include an intelligent hardware device, (e.g., a general-purpose processor, a digital signal processor (DSP), a central processing unit (CPU), a microcontroller, an application-specific integrated circuit (ASIC), a field programmable gate array (FPGA), a programmable logic device, a discrete gate or transistor logic component, a discrete hardware component, or any combination thereof). In some cases, the processor 740 may be configured to operate a memory array using a memory controller. In other cases, a memory controller may be integrated into the processor 740. The processor 740 may be configured to execute computer-readable instructions stored in a memory 735 to perform various functions (e.g., functions or tasks supporting content delivery techniques for controlling biometric parameters).

The wearable application 720 may support content delivery at the device 705 in accordance with examples as disclosed herein. For example, the wearable application 720 may be configured as or otherwise support a means for receiving biometric data associated with a user from a wearable device. The wearable application 720 may be configured as or otherwise support a means for determining that at least one biometric parameter of a set of biometric parameters associated with the biometric data satisfies a threshold during an occasion. The wearable application 720 may be configured as or otherwise support a means for selecting media content from a set of media content for recommending to the user. In some examples, each respective media content of the set of media content may be scored based at least in part on a respective effectiveness associated with each respective media content for controlling a value of the at least one biometric parameter. In some examples, the selecting may be triggered based at least in part on the at least one biometric parameter satisfying the threshold. In some examples, the media content may be selected based at least in part on a score associated with the media content. The wearable application 720 may be configured as or otherwise support a means for outputting the media content via a GUI of the device during the occasion.

By including or configuring the wearable application 720 in accordance with examples as described herein, the device 705 may support techniques for improved user experience related to reduced processing by providing media content in accordance with examples as disclosed herein.

The wearable application 720 may include an application (e.g., “app”), program, software, or other component that is configured to facilitate communications with a ring 104, server 110, other user devices 106, and the like. For example, the wearable application 720 may include an application executable on a user device 106 that is configured to receive data (e.g., physiological data) from a ring 104, perform processing operations on the received data, transmit and receive data with the servers 110, and cause presentation of data to a user 102.

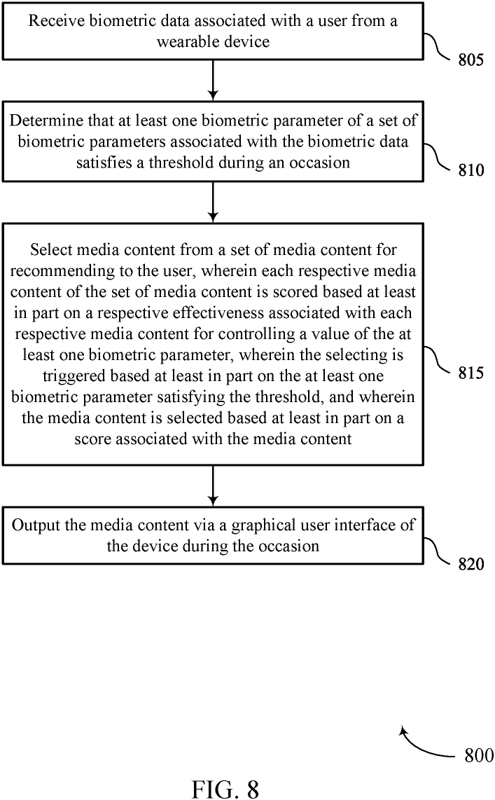

FIG. 8 shows a flowchart illustrating a method 800 that supports content delivery techniques for controlling biometric parameters in accordance with aspects of the present disclosure. The operations of the method 800 may be implemented by a user device or its components as described herein. For example, the operations of the method 800 may be performed by a user device as described with reference to FIGS. 1 through 7. In some examples, a user device may execute a set of instructions to control the functional elements of the user device to perform the described functions. Additionally, or alternatively, the user device may perform aspects of the described functions using special-purpose hardware.

At 805, the method may include receiving biometric data associated with a user from a wearable device. The operations of 805 may be performed in accordance with examples as disclosed herein. In some examples, aspects of the operations of 805 may be performed by a data module 625 as described with reference to FIG. 6.

At 810, the method may include determining that at least one biometric parameter of a set of biometric parameters associated with the biometric data satisfies a threshold during an occasion. The operations of 810 may be performed in accordance with examples as disclosed herein. In some examples, aspects of the operations of 810 may be performed by a parameter module 630 as described with reference to FIG. 6.

At 815, the method may include selecting media content from a set of media content for recommending to the user, wherein each respective media content of the set of media content is scored based at least in part on a respective effectiveness associated with each respective media content for controlling a value of the at least one biometric parameter, wherein the selecting is triggered based at least in part on the at least one biometric parameter satisfying the threshold, and wherein the media content is selected based at least in part on a score associated with the media content. The operations of 815 may be performed in accordance with examples as disclosed herein. In some examples, aspects of the operations of 815 may be performed by a media module 635 as described with reference to FIG. 6.

At 820, the method may include outputting the media content via a GUI of the device during the occasion. The operations of 820 may be performed in accordance with examples as disclosed herein. In some examples, aspects of the operations of 820 may be performed by a media module 635 as described with reference to FIG. 6.

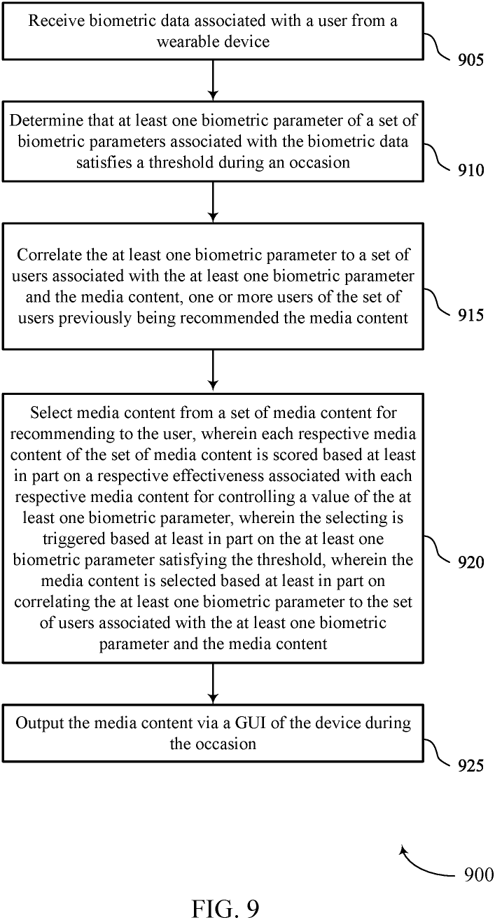

FIG. 9 shows a flowchart illustrating a method 900 that supports content delivery techniques for controlling biometric parameters in accordance with aspects of the present disclosure. The operations of the method 900 may be implemented by a user device or its components as described herein. For example, the operations of the method 900 may be performed by a user device as described with reference to FIGS. 1 through 7. In some examples, a user device may execute a set of instructions to control the functional elements of the user device to perform the described functions. Additionally, or alternatively, the user device may perform aspects of the described functions using special-purpose hardware.

At 905, the method may include receiving biometric data associated with a user from a wearable device. The operations of 905 may be performed in accordance with examples as disclosed herein. In some examples, aspects of the operations of 905 may be performed by a data module 625 as described with reference to FIG. 6.

At 910, the method may include determining that at least one biometric parameter of a set of biometric parameters associated with the biometric data satisfies a threshold during an occasion. The operations of 910 may be performed in accordance with examples as disclosed herein. In some examples, aspects of the operations of 910 may be performed by a parameter module 630 as described with reference to FIG. 6.

At 915, the method may include correlating the at least one biometric parameter to a set of users associated with the at least one biometric parameter and the media content, one or more users of the set of users previously being recommended the media content. The operations of 915 may be performed in accordance with examples as disclosed herein. In some examples, aspects of the operations of 915 may be performed by an analysis module 640 as described with reference to FIG. 6.

At 920, the method may include selecting media content from a set of media content for recommending to the user, wherein each respective media content of the set of media content is scored based at least in part on a respective effectiveness associated with each respective media content for controlling a value of the at least one biometric parameter, wherein the selecting is triggered based at least in part on the at least one biometric parameter satisfying the threshold, and wherein the media content is selected based at least in part on correlating the at least one biometric parameter to the set of users associated with the at least one biometric parameter and the media content. The operations of 920 may be performed in accordance with examples as disclosed herein. In some examples, aspects of the operations of 920 may be performed by a media module 635 as described with reference to FIG. 6.

At 925, the method may include outputting the media content via a GUI of the device during the occasion. The operations of 925 may be performed in accordance with examples as disclosed herein. In some examples, aspects of the operations of 925 may be performed by a media module 635 as described with reference to FIG. 6.

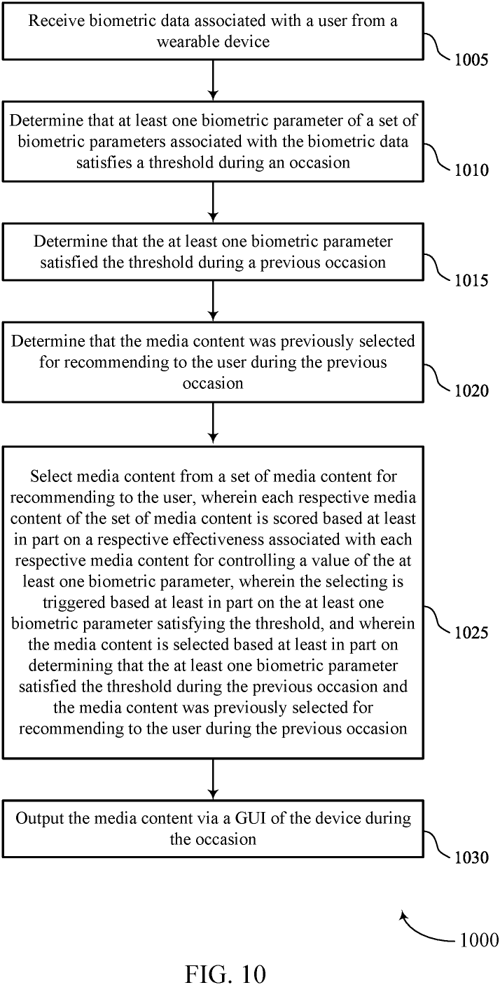

FIG. 10 shows a flowchart illustrating a method 1000 that supports content delivery techniques for controlling biometric parameters in accordance with aspects of the present disclosure. The operations of the method 1000 may be implemented by a user device or its components as described herein. For example, the operations of the method 1000 may be performed by a user device as described with reference to FIGS. 1 through 7. In some examples, a user device may execute a set of instructions to control the functional elements of the user device to perform the described functions. Additionally, or alternatively, the user device may perform aspects of the described functions using special-purpose hardware.

At 1005, the method may include receiving biometric data associated with a user from a wearable device. The operations of 1005 may be performed in accordance with examples as disclosed herein. In some examples, aspects of the operations of 1005 may be performed by a data module 625 as described with reference to FIG. 6.

At 1010, the method may include determining that at least one biometric parameter of a set of biometric parameters associated with the biometric data satisfies a threshold during an occasion. The operations of 1010 may be performed in accordance with examples as disclosed herein. In some examples, aspects of the operations of 1010 may be performed by a parameter module 630 as described with reference to FIG. 6.

At 1015, the method may include determining that the at least one biometric parameter satisfied the threshold during a previous occasion. The operations of 1015 may be performed in accordance with examples as disclosed herein. In some examples, aspects of the operations of 1015 may be performed by a parameter module 630 as described with reference to FIG. 6.

At 1020, the method may include determining that the media content was previously selected for recommending to the user during the previous occasion. The operations of 1020 may be performed in accordance with examples as disclosed herein. In some examples, aspects of the operations of 1020 may be performed by a media module 635 as described with reference to FIG. 6.

At 1025, the method may include selecting media content from a set of media content for recommending to the user, wherein each respective media content of the set of media content is scored based at least in part on a respective effectiveness associated with each respective media content for controlling a value of the at least one biometric parameter, wherein the selecting is triggered based at least in part on the at least one biometric parameter satisfying the threshold, and wherein the media content is selected based at least in part on determining that the at least one biometric parameter satisfied the threshold during the previous occasion and the media content was previously selected for recommending to the user during the previous occasion. The operations of 1025 may be performed in accordance with examples as disclosed herein. In some examples, aspects of the operations of 1025 may be performed by a media module 635 as described with reference to FIG. 6.

At 1030, the method may include outputting the media content via a GUI of the device during the occasion. The operations of 1030 may be performed in accordance with examples as disclosed herein. In some examples, aspects of the operations of 1030 may be performed by a media module 635 as described with reference to FIG. 6.

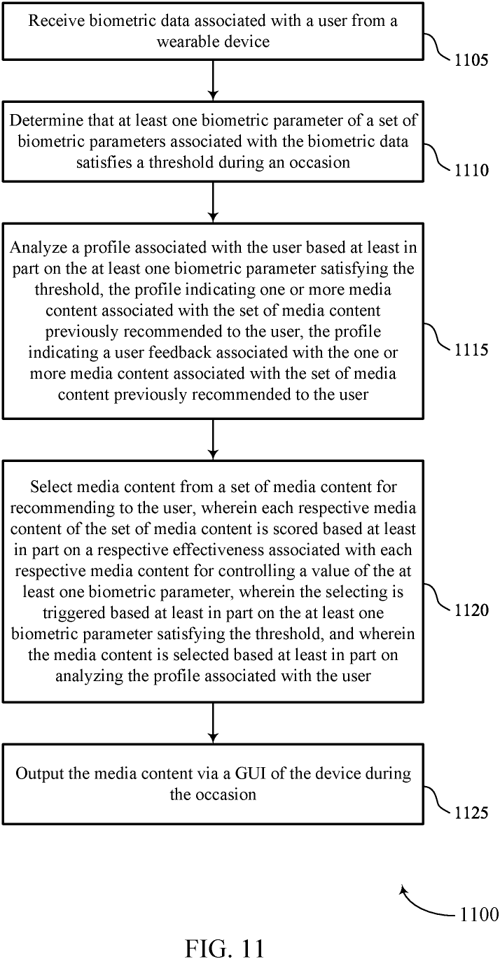

FIG. 11 shows a flowchart illustrating a method 1100 that supports content delivery techniques for controlling biometric parameters in accordance with aspects of the present disclosure. The operations of the method 1100 may be implemented by a user device or its components as described herein. For example, the operations of the method 1100 may be performed by a user device as described with reference to FIGS. 1 through 7. In some examples, a user device may execute a set of instructions to control the functional elements of the user device to perform the described functions. Additionally, or alternatively, the user device may perform aspects of the described functions using special-purpose hardware.

At 1105, the method may include receiving biometric data associated with a user from a wearable device. The operations of 1105 may be performed in accordance with examples as disclosed herein. In some examples, aspects of the operations of 1105 may be performed by a data module 625 as described with reference to FIG. 6.

At 1110, the method may include determining that at least one biometric parameter of a set of biometric parameters associated with the biometric data satisfies a threshold during an occasion. The operations of 1110 may be performed in accordance with examples as disclosed herein. In some examples, aspects of the operations of 1110 may be performed by a parameter module 630 as described with reference to FIG. 6.

At 1115, the method may include analyzing a profile associated with the user based at least in part on the at least one biometric parameter satisfying the threshold, the profile indicating one or more media content associated with the set of media content previously recommended to the user, the profile indicating a user feedback associated with the one or more media content associated with the set of media content previously recommended to the user. The operations of 1115 may be performed in accordance with examples as disclosed herein. In some examples, aspects of the operations of 1115 may be performed by a profile module 645 as described with reference to FIG. 6.

At 1120, the method may include selecting media content from a set of media content for recommending to the user, wherein each respective media content of the set of media content is scored based at least in part on a respective effectiveness associated with each respective media content for controlling a value of the at least one biometric parameter, wherein the selecting is triggered based at least in part on the at least one biometric parameter satisfying the threshold, and wherein the media content is selected based at least in part on analyzing the profile associated with the user. The operations of 1120 may be performed in accordance with examples as disclosed herein. In some examples, aspects of the operations of 1120 may be performed by a media module 635 as described with reference to FIG. 6.

At 1125, the method may include outputting the media content via a GUI of the device during the occasion. The operations of 1125 may be performed in accordance with examples as disclosed herein. In some examples, aspects of the operations of 1125 may be performed by a media module 635 as described with reference to FIG. 6.

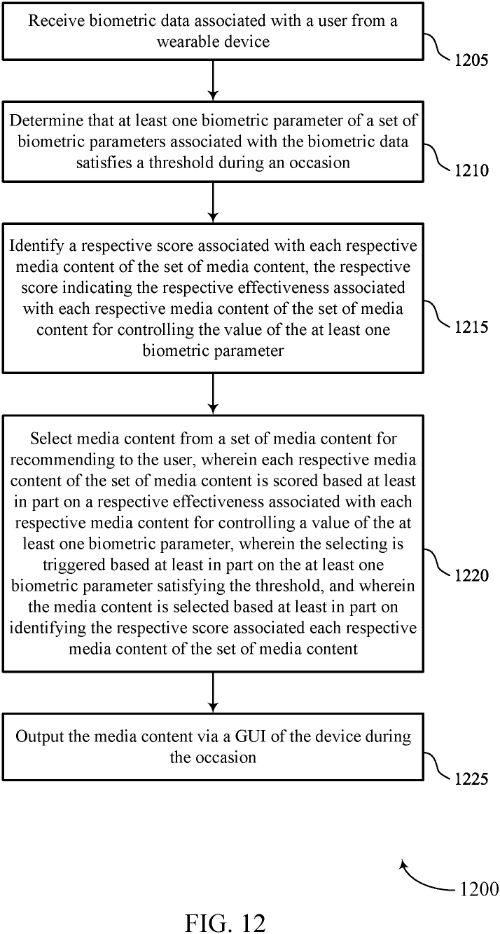

FIG. 12 shows a flowchart illustrating a method 1200 that supports content delivery techniques for controlling biometric parameters in accordance with aspects of the present disclosure. The operations of the method 1200 may be implemented by a user device or its components as described herein. For example, the operations of the method 1200 may be performed by a user device as described with reference to FIGS. 1 through 7. In some examples, a user device may execute a set of instructions to control the functional elements of the user device to perform the described functions. Additionally, or alternatively, the user device may perform aspects of the described functions using special-purpose hardware.

At 1205, the method may include receiving biometric data associated with a user from a wearable device. The operations of 1205 may be performed in accordance with examples as disclosed herein. In some examples, aspects of the operations of 1205 may be performed by a data module 625 as described with reference to FIG. 6.

At 1210, the method may include determining that at least one biometric parameter of a set of biometric parameters associated with the biometric data satisfies a threshold during an occasion. The operations of 1210 may be performed in accordance with examples as disclosed herein. In some examples, aspects of the operations of 1210 may be performed by a parameter module 630 as described with reference to FIG. 6.

At 1215, the method may include identifying a respective score associated with each respective media content of the set of media content, the respective score indicating the respective effectiveness associated with each respective media content of the set of media content for controlling the value of the at least one biometric parameter. The operations of 1215 may be performed in accordance with examples as disclosed herein. In some examples, aspects of the operations of 1215 may be performed by a score module 655 as described with reference to FIG. 6.

At 1220, the method may include selecting media content from a set of media content for recommending to the user, wherein each respective media content of the set of media content is scored based at least in part on a respective effectiveness associated with each respective media content for controlling a value of the at least one biometric parameter, wherein the selecting is triggered based at least in part on the at least one biometric parameter satisfying the threshold, and wherein the media content is selected based at least in part on identifying the respective score associated each respective media content of the set of media content. The operations of 1220 may be performed in accordance with examples as disclosed herein. In some examples, aspects of the operations of 1220 may be performed by a media module 635 as described with reference to FIG. 6.

At 1225, the method may include outputting the media content via a GUI of the device during the occasion. The operations of 1225 may be performed in accordance with examples as disclosed herein. In some examples, aspects of the operations of 1225 may be performed by a media module 635 as described with reference to FIG. 6.

It should be noted that the methods described above describe possible implementations, and that the operations and the steps may be rearranged or otherwise modified and that other implementations are possible. Furthermore, aspects from two or more of the methods may be combined.

The following provides an overview of aspects of the present disclosure:
Aspect 1: A method for content delivery at a device, comprising: receiving biometric data associated with a user from a wearable device; determining that at least one biometric parameter of a set of biometric parameters associated with the biometric data satisfies a threshold during an occasion; selecting media content from a set of media content for recommending to the user, wherein each respective media content of the set of media content is scored based at least in part on a respective effectiveness associated with each respective media content for controlling a value of the at least one biometric parameter, wherein the selecting is triggered based at least in part on the at least one biometric parameter satisfying the threshold, and wherein the media content is selected based at least in part on a score associated with the media content; and outputting the media content via a GUI of the device during the occasion.
Aspect 2: The method of aspect 1, further comprising: correlating the at least one biometric parameter to a set of users associated with the at least one biometric parameter and the media content, one or more users of the set of users previously being recommended the media content, wherein selecting the media content is further based at least in part on correlating the at least one biometric parameter to the set of users associated with the at least one biometric parameter and the media content.
Aspect 3: The method of any of aspects 1 through 2, further comprising: determining that the at least one biometric parameter satisfied the threshold during a previous occasion; and determining that the media content was previously selected for recommending to the user during the previous occasion, wherein selecting the media content is further based at least in part on determining that the at least one biometric parameter satisfied the threshold during the previous occasion and the media content was previously selected for recommending to the user during the previous occasion.
Aspect 4: The method of any of aspects 1 through 3, further comprising: analyzing a profile associated with the user based at least in part on the at least one biometric parameter satisfying the threshold, the profile indicating one or more media content associated with the set of media content previously recommended to the user, the profile indicating a user feedback associated with the one or more media content associated with the set of media content previously recommended to the user, wherein selecting the media content is further based at least in part on analyzing the profile associated with the user.
Aspect 5: The method of any of aspects 1 through 4, further comprising: determining an activity associated with the user during the occasion; correlating the activity associated with the user to one or more of the at least one biometric parameter and the media content, wherein selecting the media content is further based at least in part on the correlating the activity associated with the user to one or more of the at least one biometric parameter and the media content.
Aspect 6: The method of any of aspects 1 through 5, further comprising: identifying a respective score associated with each respective media content of the set of media content, the respective score indicating the respective effectiveness associated with each respective media content of the set of media content for controlling the value of the at least one biometric parameter, wherein selecting the media content is further based at least in part on identifying the respective score associated each respective media content of the set of media content.
Aspect 7: The method of any of aspects 1 through 6, further comprising: receiving an input from the user via the GUI of the device; enabling the media content for a duration associated with the media content based at least in part on receiving the input from the user via the GUI of the device; and monitoring for biometric feedback data associated with the user from the wearable device during the duration associated with the media content based at least in part on the enabling.
Aspect 8: The method of aspect 7, further comprising: receiving the biometric feedback data associated with the user from the wearable device during the duration associated with the media content based at least in part on the monitoring; determining a biometric change to the at least one biometric parameter of the set of biometric parameters associated with the biometric data based at least in part on the biometric feedback data associated with the user; and outputting a representation of the biometric change to the biometric data via the GUI of the device.
Aspect 9: The method of any of aspects 1 through 8, further comprising: assigning a rank to the media content for recommending to the user during a subsequent occasion and for ordering the media content in a list of media content provided to the user via the GUI of the device based at least in part on the biometric change to the at least one biometric parameter of the set of biometric parameters associated with the biometric data.
Aspect 10: The method of any of aspects 1 through 9, wherein the media content comprises audio content.
Aspect 11: The method of any of aspects 1 through 10, wherein the at least one biometric parameter of the set of biometric parameters associated with the biometric data comprises heart rate data associated with the user, respiratory rate data associated with the user, sleep data associated with the user, activity data associated with the user, or any combination thereof.
Aspect 12: The method of any of aspects 1 through 11, wherein the wearable device comprises a wearable ring device.
Aspect 13: An apparatus for content delivery at a device, comprising a processor; memory coupled with the processor; and instructions stored in the memory and executable by the processor to cause the apparatus to perform a method of any of aspects 1 through 12.
Aspect 14: An apparatus for content delivery at a device, comprising at least one means for performing a method of any of aspects 1 through 12.
Aspect 15: A non-transitory computer-readable medium storing code for content delivery at a device, the code comprising instructions executable by a processor to perform a method of any of aspects 1 through 12.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (e.g., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for content delivery at a device, comprising:
receiving biometric data associated with a user from a wearable device;
determining that at least one biometric parameter of a set of biometric parameters associated with the biometric data satisfies a threshold during an occasion;
selecting media content from a set of media content for recommending to the user, wherein each respective media content of the set of media content is scored based at least in part on a respective effectiveness associated with each respective media content for controlling a value of the at least one biometric parameter, wherein the selecting is triggered based at least in part on the at least one biometric parameter satisfying the threshold, and wherein the media content is selected based at least in part on a score associated with the media content; and
outputting the media content via a graphical user interface of the device during the occasion.

2. The method of claim 1, further comprising:
correlating the at least one biometric parameter to a set of users associated with the at least one biometric parameter and the media content, one or more users of the set of users previously being recommended the media content,
wherein selecting the media content is further based at least in part on correlating the at least one biometric parameter to the set of users associated with the at least one biometric parameter and the media content.

3. The method of claim 1, further comprising:
determining that the at least one biometric parameter satisfied the threshold during a previous occasion; and
determining that the media content was previously selected for recommending to the user during the previous occasion,
wherein selecting the media content is further based at least in part on determining that the at least one biometric parameter satisfied the threshold during the previous occasion and the media content was previously selected for recommending to the user during the previous occasion.

4. The method of claim 1, further comprising:
analyzing a profile associated with the user based at least in part on the at least one biometric parameter satisfying the threshold, the profile indicating one or more media content associated with the set of media content previously recommended to the user, the profile indicating a user feedback associated with the one or more media content associated with the set of media content previously recommended to the user,
wherein selecting the media content is further based at least in part on analyzing the profile associated with the user.

5. The method of claim 1, further comprising:
determining an activity associated with the user during the occasion; and
correlating the activity associated with the user to one or more of the at least one biometric parameter and the media content,
wherein selecting the media content is further based at least in part on the correlating the activity associated with the user to one or more of the at least one biometric parameter and the media content.

6. The method of claim 1, further comprising:
identifying a respective score associated with each respective media content of the set of media content, the respective score indicating the respective effectiveness associated with each respective media content of the set of media content for controlling the value of the at least one biometric parameter,
wherein selecting the media content is further based at least in part on identifying the respective score associated each respective media content of the set of media content.

7. The method of claim 1, further comprising:
receiving an input from the user via the graphical user interface of the device;
enabling the media content for a duration associated with the media content based at least in part on receiving the input from the user via the graphical user interface of the device; and
monitoring for biometric feedback data associated with the user from the wearable device during the duration associated with the media content based at least in part on the enabling.

8. The method of claim 7, further comprising:
receiving the biometric feedback data associated with the user from the wearable device during the duration associated with the media content based at least in part on the monitoring;
determining a biometric change to the at least one biometric parameter of the set of biometric parameters associated with the biometric data based at least in part on the biometric feedback data associated with the user; and
outputting a representation of the biometric change to the biometric data via the graphical user interface of the device.

9. The method of claim 1, further comprising:
assigning a rank to the media content for recommending to the user during a subsequent occasion and for ordering the media content in a list of media content provided to the user via the graphical user interface of the device based at least in part on the biometric change to the at least one biometric parameter of the set of biometric parameters associated with the biometric data.

10. The method of claim 1, wherein the media content comprises audio content or video content.

11. The method of claim 1, wherein the at least one biometric parameter of the set of biometric parameters associated with the biometric data comprises heart rate data associated with the user, respiratory rate data associated with the user, sleep data associated with the user, activity data associated with the user, or any combination thereof.

12. The method of claim 1, wherein the wearable device comprises a wearable ring device.

13. An apparatus for content delivery, comprising:
a processor;
memory coupled with the processor; and
instructions stored in the memory and executable by the processor to cause the apparatus to:
receive biometric data associated with a user from a wearable device;
determine that at least one biometric parameter of a set of biometric parameters associated with the biometric data satisfies a threshold during an occasion;
select media content from a set of media content for recommending to the user, wherein each respective media content of the set of media content is scored based at least in part on a respective effectiveness associated with each respective media content for controlling a value of the at least one biometric parameter, wherein the selecting is triggered based at least in part on the at least one biometric parameter satisfying the threshold, and wherein the media content is selected based at least in part on a score associated with the media content; and
output the media content via a graphical user interface of the apparatus during the occasion.

14. The apparatus of claim 13, wherein the instructions are further executable by the processor to cause the apparatus to:
correlate the at least one biometric parameter to a set of users associated with the at least one biometric parameter and the media content, one or more users of the set of users previously being recommended the media content,
wherein the instructions to select the media content are further executable by the processor based at least in part on correlating the at least one biometric parameter to the set of users associated with the at least one biometric parameter and the media content.

15. The apparatus of claim 13, wherein the instructions are further executable by the processor to cause the apparatus to:
determine that the at least one biometric parameter satisfied the threshold during a previous occasion; and
determine that the media content was previously selected for recommending to the user during the previous occasion,
wherein the instructions to select the media content are further executable by the processor based at least in part on determining that the at least one biometric parameter satisfied the threshold during the previous occasion and the media content was previously selected for recommending to the user during the previous occasion.

16. The apparatus of claim 13, wherein the instructions are further executable by the processor to cause the apparatus to:
analyze a profile associated with the user based at least in part on the at least one biometric parameter satisfying the threshold, the profile indicating one or more media content associated with the set of media content previously recommended to the user, the profile indicating a user feedback associated with the one or more media content associated with the set of media content previously recommended to the user,
wherein the instructions to select the media content are further executable by the processor based at least in part on analyzing the profile associated with the user.

17. The apparatus of claim 13, wherein the instructions are further executable by the processor to cause the apparatus to:
determine an activity associated with the user during the occasion; and
correlate the activity associated with the user to one or more of the at least one biometric parameter and the media content,
wherein the instructions to select the media content are further executable by the processor based at least in part on the correlating the activity associated with the user to one or more of the at least one biometric parameter and the media content.

18. The apparatus of claim 13, wherein the instructions are further executable by the processor to cause the apparatus to:
identify a respective score associated with each respective media content of the set of media content, the respective score indicating the respective effectiveness associated with each respective media content of the set of media content for controlling the value of the at least one biometric parameter,
wherein the instructions to select the media content are further executable by the processor based at least in part on identifying the respective score associated each respective media content of the set of media content.

19. The apparatus of claim 13, wherein the instructions are further executable by the processor to cause the apparatus to:
receive an input from the user via the graphical user interface of the apparatus;
enable the media content for a duration associated with the media content based at least in part on receiving the input from the user via the graphical user interface of the apparatus; and
monitor for biometric feedback data associated with the user from the wearable device during the duration associated with the media content based at least in part on the enabling.

20. An apparatus for content delivery, comprising:
means for receiving biometric data associated with a user from a wearable device;
means for determining that at least one biometric parameter of a set of biometric parameters associated with the biometric data satisfies a threshold during an occasion;
means for selecting media content from a set of media content for recommending to the user, wherein each respective media content of the set of media content is scored based at least in part on a respective effectiveness associated with each respective media content for controlling a value of the at least one biometric parameter, wherein the selecting is triggered based at least in part on the at least one biometric parameter satisfying the threshold, and wherein the media content is selected based at least in part on a score associated with the media content; and
means for outputting the media content via a graphical user interface of the apparatus during the occasion.
